(19) United States
(12) Patent Application Publication
(10) Pub. No.: US 2012/0245088 A1
(43) Pub. Date: Sep. 27, 2012

(54) COMPOSITIONS COMPRISING IGF1 AGONISTS AND USES THEREOF

(76) Inventor: Kenneth W. Adams, North York (CA)

(21) Appl. No.: 13/513,634

(22) PCT Filed: Dec. 3, 2010
(86) PCT No.: PCT/CA10/01925

§ 371 Date: Jun. 4, 2012

(30) Foreign Application Priority Data
Dec. 3, 2009 (CA) 2687012

(51) Int. Cl. A61K 38/30; A61Q 19/08; A61Q 19/00; A61P 17/00; A61P 9/00; A61P 19/04; A61P 19/08; A61P 21/00; A61K 8/64; A61P 17/14

(52) U.S. Cl. 514/8.6

(57) ABSTRACT

Pharmaceutical compositions and methods for improving functional capacity of tissues and organs in both healthy and pathologic disease states for: reducing unwanted cosmetic effects of aging on the skin to reduce wrinkles, to increase collagen and water content, thicken and increase skin volume and to maintain a youthful appearance to age and sun damaged skin, to treat diseases of skin and tissues caused by ischemia of venous congestion and arterial insufficiency, to accelerate healing of tendons, ligaments, joints, cartilage, nerves, bones or neuromuscular injuries or diseases, to improve and restore the functional capacity of specific tissues and organs of the body, and to treat hair loss comprising an effective amount of an insulin-like growth factor selected from the group consisting of IGF-1 (Somatmedin-C) and analogue LR3 IGF1 in admixture with a pharmaceutically-acceptable diluent or carrier.

### FIELD OF THE INVENTION

The invention relates to methods and compositions for reducing the unwanted cosmetic effects of aging on the skin by reducing wrinkles, increasing collagen, activating stem cells, restoring volume and water content thereby restoring a youthful appearance to age and sun damaged skin; for the treatment of hair loss, particularly, reversing the cosmetic effects of hair loss or alternatively, for thickening the hair of individuals with naturally fine hair; for accelerating healing and recovery for Neuromuscular degenerative disorders; for accelerating healing and repair of tendons, ligaments, and osseous fractures; and for accelerating revascularization and healing for medical conditions associated with impaired tissue perfusion for a wide range of conditions including venous and arterial vascular diseases.

### BACKGROUND OF THE INVENTION

The demand for cosmetic surgical procedures to reduce wrinkles and age related changes in the skin of men and women have increased dramatically in recent years. These surgical procedures do not undo the loss of collagen, or the reduction in the molecular and cellular turnover that are associated with aged and sun damaged skin. Further, the limitations of existing surgical treatments such as BOTOX and fillers such as Perlane and Restylane can only be used in limited areas. Other procedures using laser and other energy modalities have been developed to promote improved skin.

There are no proven pharmacological treatments that can actually reverse or slow cellular and biochemical changes that result in a significant reduction in the volume and loss of skin elasticity and increased wrinkles and sagging of old skin.

There is, therefore, a need for an alternative method to the aforesaid surgical techniques to enhance the cosmetic appearance of skin.

One such method would be the safe, preferably, local application of suitable efficacious pharmaceutical/pharmacological agents to reverse skin related aging and sun damage, reduce wrinkles and to restore a more youthful cellular and biochemical turnover in the skin and subcutaneous tissues of the face and skin in other areas of the body. This would be most cosmetically desirable for both men and women.

The demand for cosmetic hair transplants procedures to treat hair loss in men and women have increased dramatically in recent years. And even though large numbers of women are significantly impacted by hair loss, the majority of women are not good candidates since the traditional donor areas are often thinned and depleted in females with noticeable thinning, resulting in often inadequate donor supply. As well women concerned about hair loss and thinning often have much thicker hair than males seeking this cosmetic surgical treatment, which frequently requires more hair to be transplanted than can be harvested to yield a significant cosmetic improvement. Thus the majority of women who might consider a hair transplant would be rejected by surgeons skilled in the art as they would be considered poor surgical candidates. As a result, women only with severe hair loss similar to a male type pattern of loss can expect a significant improvement to justify a surgical hair transplant.

Proven pharmacological treatment can delay or slow further hair loss and increase hair density in the vertex in some cases. This would include topical medications such as Minoxidil and systemic medication such as Finesteride and Dutesteride, but unfortunately these medications have been shown to have limited effects only in the vertex area at the back of the head, and they do not have any significant effect in the frontal hair line, which is an area that is of the most importance to the majority of people experiencing hair loss.

And since up to 25-50% of adults have a cosmetically significant thinning or reduction in hair density as they age, hair loss has a very significant impact on society. As well, some individuals are born with naturally thin fine hair which is especially of significant cosmetic concern.

Due to the limitations of the existing medical and surgical options, other treatment options including toupees and hair systems, as well as other procedures using laser, and other energy modalities have been developed to promote improved hair growth.

Musculoskeletal injuries to hard (bone and cartilage) and soft tissues (tendons, ligaments, fascia, muscle, synovial tissues) as treated generally by immobilization and rest followed by graduated activities such as physiotherapy and exercise to achieve optimal rates of healing, But prior to this invention there have been no pharmacological agents available to speed up or accelerate healing, or to produce improved clinical outcomes.

Neuromuscular injuries and functional neuromuscular impairments have traditionally been treated by elimination of behavioral and environmental factors that may have precipitated the decline in function, followed by periods of healing. Medications for pain or inflammation are prescribed to relieve symptoms, but until this invention there were no therapeutic pharmacologic agents available to accelerate healing or to improve the functional capacity of damaged or declining tissues or organs to produce improved clinical outcomes.

Traumatic injuries and functional impairments to tissues and organs have traditionally been treated by elimination of behavioral and environmental factors that may have precipitated the decline in function, followed by periods of healing and rehabilitation. Medications for pain or inflammation are prescribed to relieve symptoms, but until this invention was discovered by the inventor there were no therapeutic pharmacologic agents available to accelerate healing or to improve the functional capacity of damaged or declining tissues or organs to produce improved clinical outcomes.

The functional declines associated with normal and pathological (accelerated or premature) aging may now be addressed. Potential local applications could eliminate congestive heart failure in patients with impaired cardiac function, improve glomerular filtration rates and renal function in patients with declining renal function, improve hepatic function in patients with cirrhosis, or other forms of hepatic disease associated with a loss of functional hepatic tissue especially when associated with signs and symptoms of impaired hepatic function.

### SUMMARY OF THE INVENTION

The invention relates to methods and composition that improves the functional capacity of tissues and organs in both healthy and pathologic disease states for: cosmetic restoration of volume of aged skin to reverse the molecular and cellular changes associated with aging of the skin to visibly reduce wrinkles, increase collagen and other molecular elements of epidermis, dermis and subdermis. By increasing cellular activity while inhibiting apoptosis these therapeutic agents will increase molecular and cellular elements which can expand volume and water content thereby restoring a youthful appearance to aged and sun damaged skin; for the treatment of hair loss, particularly, reversing the cosmetic effects of hair loss or alternatively, for thickening the hair of individuals with naturally fine hair or to enhance the density and thickness of hair transplant procedures; for accelerating healing and recovery following function neuronal loss or denervation following injuries (traumatic, chemical, drug, inflammatory, autoimmune) or other pathological diseases of both the central or peripheral nervous system; neuromuscular degenerative disorders; for accelerating healing and repair of tendons, ligaments, and osseous fractures; and accelerating revascularization and healing for medical conditions associated with impaired tissue perfusion for a wide range of conditions including venous and arterial vascular diseases.

In its broadest aspect, the invention provides pharmaceutical compositions applied locally or systemically that will improve functional capacity of tissues and organs in both healthy and pathologic disease states. This would include primarily local or possibly systemic application to the skin of the face and possibly other areas of the body as a methods for reducing unwanted cosmetic effects of aging on the skin to reduce wrinkles, to increase collagen and water content, thicken and increase skin volume and to maintain a youthful appearance to age and sun damaged skin, to treat diseases of skin and tissues caused by ischemic of venous congestion and arterial insufficiency, to accelerate healing or induce improved functional capacity of organs such as the heart, liver, kidney, thyroid gland, adrenal gland, gonads, ovaries, etc and to improve the functional capacity of tissues such as tendons, ligaments, joints, cartilage, nerves, bones or neuromuscular injuries or diseases and to alleviate hair loss comprising an effective amount of an insulin-like growth factor selected from the group consisting of IGF-1 (Somatmedin-C) and analogue LR3 IGF1 in admixture with a pharmaceutically-acceptable diluent or carrier.

Following any traumatic injury to the body, the cellular repair mechanisms of the body in elderly, malnourished and chronically ill individuals are declining and the need for accelerated healing is increased. The localized delivery and use of IGF1 receptor agonists in localized areas can increase stem cell activities and increase the secretion of other growth and reparative cytokines to restore tissues to a more youthful state, and in diseased or injured tissues, they can accelerate healing and reduce recovery times.

One example of an application of IGF1 agonists discovered by the inventor shows dramatic recovery of strength and function in de-enervated muscles years after the initial injury, and after a prolonged period of stability with no improvement, there was dramatic and significant recovery.

Further examples demonstrate the benefits of IGF1 agonists to accelerate the healing and strengthening that occurs after the rupture or tearing of a tendon or ligament but these effects also apply to muscle, cartilage, bone and other connective tissues.

Yet further examples demonstrate the benefits of IGF1 agonists to accelerate the healing and restoration of health in association with ischemia in venous and arterial disease. The inventor has observed and discovered numerous benefits from the localized actions of IGF1 agonists to accelerate healing especially in diseased and aged tissues to restore them to a healthier, more youthful state.

In one aspect, the present invention provides a method and composition for reducing unwanted cosmetic effects of aging on the skin to reduce wrinkles, to increase collagen and water content, thicken and increase skin volume and to restore a youthful appearance to age and sun damaged skin, and alleviate hair loss.

The inventor has observed that by the application of IGF1 Agonist or PGF2 or Bimatoprost, these agents can thicken all types of hair in the scalp, (or eyebrows or eyelashes) that have undergone miniaturization but are still present. Furthermore, PGF2alpha has been observed to thicken healthy hairs and can thus even thicken and significantly increase the hair density in patients without hair loss. These agents appear to be able to thicken hair in the frontal hair line that does not respond to the currently used pharmacological treatments of Minoxidil and the 5alpha-reductase blockers Finesteride and Dutesterisde. As well, it can be applied locally to thicken eyebrows and to thicken eyelashes which is especially of interest to many women and some men.

As well, IGF1 locally applied to the scalp, either alone or topically or by injecting to the scalp, has resulted in increased hair growth in some patients. As well IGF1 can be combined with Prostaglandins such as PGF2, PGE2 and PGE1 for even better effect.

As a result, the safe local application of pharmacological agents to increase hair growth, hair thickness and hair density are cosmetically desirable for both men and women.

Accordingly, in one aspect the invention provides a pharmaceutical composition for reducing unwanted cosmetic effects of aging on the skin to reduce wrinkles, to increase collagen and water content, thicken and increase skin volume and to maintain a youthful appearance to age and sun damaged skin and alleviate hair loss, said composition comprising an effective amount of an insulin-like growth factor selected from the group consisting of IGF-1 and LR3 1GF1 and a pharmaceutically-acceptable carrier. And the inventor has observed

In a further aspect, the invention provides a pharmaceutical composition for accelerating revascularization and healing for medical conditions associated with impaired tissue perfusion for a wide range of conditions including venous and arterial vascular diseases comprising an effective amount of an insulin-like growth factor selected from the group consisting of IGF-1 (Somatomedin-C) and analogue LR3 IGF1 in admixture with a pharmaceutically-acceptable diluent.

In a further aspect, the invention provides a pharmaceutical composition for accelerated healing and recovery for neuromuscular degenerative disorders comprising an effective amount of an insulin-like growth factor selected from the group consisting of IGF-1 (Somatomedin-C) and analogue LR3 IGF1 in admixture with a pharmaceutically-acceptable diluent.

In a further aspect, the invention provides a pharmaceutical composition for accelerated healing and recovery for traumatic or degenerative disorders of different tissues or organs comprising an effective amount of an insulin-like growth factor selected from the group consisting of IGF-1 (Somatomedin-C) and analogue LR3 IGF1 in admixture with a pharmaceutically-acceptable diluent.

In a further aspect, the invention provides a pharmaceutical composition for accelerated healing and repair of tendons, ligaments, and osseous fractures comprising an effective amount of an insulin-like growth factor selected from the group consisting of IGF-1 (Somatomedin-C) and analogue LR3 IGF1 in admixture with a pharmaceutically-acceptable diluent.

IGF-I is also known as Somatomedin-C which comprises a full length protein of 70 amino acids in a single chain with three intramolecular disulfide bridges and LR3IGF1 is an analogue of IGF-I. Examples of recombinant human IGF-I and recombinant LR3 IGF-I are products made by GroPep Ltd. (Adelaide, Australia) and other commercial laboratories.

In preferred embodiments, the compositions hereinabove defined further comprise a prostaglandin, more preferably, prostaglandins selected from PGE1 and PGF2.

In a further aspect, the invention provides a method for reducing unwanted cosmetic effects of aging on the skin to reduce wrinkles, to increase collagen and water content, thicken and increase skin volume and to maintain a youthful appearance to age and sun-damaged skin, said method comprising applying a composition as hereinabove defined to the selected skin tissue. When IGF-I and recombinant LR3 IGF-I were premixed and compounded with a low viscosity Hyaluronic Acid it seemed produce a synergistic effect. The Hyaluronic acid acid seemed to trap the IGF-I and recombinant LR3 IGF-I to yield a sustained and enhanced effect appeared to result in dramatic ingrowth of cellular elements that yielded a much more prolonged duration of effect of the combined IGF-I with Hyaluronic Acid or the LR3 IGF-I combined with Hyaluronic Acid. The combined effect was clearly superior to the Hyaluronic Acid alone. In a further aspect, the invention provides a method for accelerating revascularization and healing for medical conditions associated with impaired tissue perfusion for a wide range of conditions including venous and arterial vascular diseases, said method comprising administering to a human a composition comprising an effective amount of an insulin-like growth factor selected from the group consisting of IGF-1 (Somatomedin-C) and analogue LR3 IGF1 in admixture with a pharmaceutically-acceptable diluent.

In a further aspect, the invention provides a method for accelerated healing and recovery for Neuromuscular degenerative disorders comprising administering to a human a composition comprising an effective amount of an insulin-like growth factor selected from the group consisting of IGF-1 (Somatomedin-C) and analogue LR3 IGF1 in admixture with a pharmaceutically-acceptable diluent.

In a further aspect, the invention provides a pharmaceutical composition for accelerated healing and repair of tendons, ligaments, osseous fractures comprising an effective amount of an insulin-like growth factor selected from the group consisting of IGF-1 (Somatomedin-C) and analogue LR3 IGF1 in admixture with a pharmaceutically-acceptable diluent.

In a preferred practice of the invention, the compositions of IGF-1 or LR3 IGF-I of use according to the invention are administered, for example, by subcutaneous injection, high pressure jet device, intracavernous injection, intravenous injection, intramuscular injection, intradermal injection, intra-nasal or topical administration with or without a prostaglandin in admixture. Alternatively the prostaglandin may be applied, independently, by injection or it may be effectively applied, topically, as a spray, cream, gel or patch at a dose selected from 0.1 to 100 μg.

In a further aspect, the invention provides a method of manufacturing a medicament intended for the application of anti-aging as hereinabove described characterized in that insulin-like growth factor (IGF) selected from IGF-1 (Somatomedin-C) and LR3 IGF1 is admixed with a pharmaceutically-acceptable carrier.

In a further aspect, the invention provides a method of manufacturing a medicament intended for accelerating revascularization and healing for medical conditions associated with impaired tissue perfusion for a wide range of conditions including venous and arterial vascular diseases characterized in that insulin-like growth factor (IGF) selected from IGF-1 (Somatomedin-C) and LR3 IGF1 is admixed with a pharmaceutically-acceptable carrier.

In a further aspect, the invention provides a method of manufacturing a medicament intended for accelerated healing and recovery for Neuromuscular degenerative disorders characterized in that insulin-like growth factor (IGF) selected from IGF-1 (Somatomedin-C) and LR3 IGF1 is admixed with a pharmaceutically-acceptable carrier.

In a further aspect, the invention provides a method of manufacturing a medicament intended for accelerated healing and repair of tendons, ligaments, osseous fractures characterized in that insulin-like growth factor (IGF) selected from IGF-1 (Somatomedin-C) and LR3 IGF1 is admixed with a pharmaceutically-acceptable carrier.

In a further aspect, the invention provides use of a composition comprising an effective amount of insulin-like growth factor selected from the group consisting of IGF-I and LR3 IGFI and a pharmaceutically-acceptable carrier for reducing unwanted effects of aging on the skin to reduce wrinkles, to increase collagen and water content, thicken and increase skin volume and to maintain a youthful appearance to aged and sun-damaged skin.

In a further aspect, the invention provides use of a pharmaceutical composition for accelerating revascularization and healing for medical conditions associated with impaired tissue perfusion for a wide range of conditions including venous and arterial vascular diseases, said composition comprising an effective amount of an insulin-like growth factor selected from the group consisting of IGF-1 (Somatomedin-C) and analogue LR3 IGF1 in admixture with a pharmaceutically-acceptable diluent.

In a further aspect, the invention provides use of a pharmaceutical composition for accelerated healing and recovery for Neuromuscular degenerative disorders, said composition comprising an effective amount of an insulin-like growth factor selected from the group consisting of IGF-1 (Somatomedin-C) and analogue LR3 IGF1 in admixture with a pharmaceutically-acceptable diluent.

In a further aspect, the invention provides use of a pharmaceutical composition for accelerated healing and repair of tendons, ligaments, osseous fractures, said composition comprising an effective amount of an insulin-like growth factor selected selected from the group consisting of IGF-1 (Somatomedin-C) and analogue LR3 IGF1 in admixture with a pharmaceutically-acceptable diluent.

Preferably, the composition comprises a prostaglandin, more preferably, selected from prostaglandin E1 and prostaglandin F1.

Administration to the skin tissue also encompasses injections to the connective tissues. Injections may be performed by various methods including by needle, auto-injector, slow-sustained injection pump or micro-pump, high-pressure injection device, and micro infusion pump. Other routes of administration to the tissue include implantable sustained-release drugs or devices, and transdermal devices or vehicles which are directly in contact with or adhered to the skin, such as patches, creams, or lotions. The active ingredient can also be delivered transdermally. For prostaglandins they are well absorbed topically. IGF1 agonists are proteins that do not absorb well transdermally and are best applied by parenteral routes. A suitable delivery vehicle or device is situated in direct contact with the skin to effect delivery of the active ingredient. The vehicle or device may include agents which enhance the transdermal absorption rate or agents which aid in the absorption of the pharmaceutical composition into the tissue.

A preferred transdermal device of the present invention is a patch. A patch is designed to adhere to or be brought into contact with the skin so that the pharmaceutical composition contained by the patch can be absorbed transdermally. The patch may also contain agents which enhance, control, or a combination of both, the transdermal absorption of the pharmaceutical composition and/or the absorption of the pharmaceutical composition into the skin. Optionally, these agents can be applied in conjunction with the pharmaceutical composition, at a different time, and/or a different route of administration. The patch may also include adhesives specially designed to adhere to sensitive skin.

Pharmaceutical compositions of the present invention which are given as injections may be injected in an oil-based carrier (depot oil) or other sustained release formulation into the skin tissue or into the connective tissue which surrounds the skin tissue.

Water-based pharmaceutical compositions or emulsifications injected into the skin tissue are also effective. As well, transdermal devices or vehicles such as patches may include agents, which enhance the transdermal absorption rate or agents which aid in the absorption of the pharmaceutical composition into the skin tissue.

Kits comprising pharmaceutical compositions of the invention formulated in sterile unit dosage forms suitable for administration to the adipose tissue, and which include instructions for use in written, oral, videotape, compact disc, other digital electronic form, or other recorded media, are contemplated. Conveniently, a kit wherein the sterile unit dosage forms are for oil based depot injections and instructions for use are provided.

Those of skill in the art will understand that the increased percentage absorption of the active agent of the pharmaceutical compositions of the present invention into the tissue compared to that achieved from the usual administered systemic dose is because of the different route of administration.

The appropriate dosage and frequency of treatment may vary depending upon desire or need of the degree of cosmetic improvement sought. Other health related factors would also be considered.

The frequency of administration may be in the range of two to three times a day by injection, more preferably from 3 to 7 times a week, or as infrequently as monthly for delayed release formulations or topical administrations.

Thus, the present invention provides for methods, compositions, uses and kits for the treatments as hereinabove defined, which involves the administration of insulin-like growth factors and optionally prostaglandins as hereinabove defined.

In a further aspect, the invention provides for a kit comprising the above described compositions and an instruction for using the compositions for the purpose as hereinabove defined.

### DETAILED DESCRIPTION OF PREFERRED EMBODIMENTS

In order that the invention may be better understood, preferred embodiments will now be described by way of example only with reference to the following examples.

### EXAMPLE 1

A 53 year old male was initially injected with 0.05 ml of IGF-1 (50 μg/ml) at each of three points at lateral and inferior locations adjacent the right and left eyes showing crow's-feet wrinkles.

A noticeable reduction in the wrinkles was noticed after 3 days. After 5 days, there was a further reduction in the wrinkles and a clear increase in the skin volume.

After 2 weeks the improvement persisted and further repeat injections of the same dose were made at each of the aforesaid three points and, in addition, 0.05 ml of IGF-I (50 μg/ml) doses were injected at each of two points to deep forehead wrinkles.

After 3 days there was a noticeable improvement in reduction of the forehead wrinkles and a continued reduction in the R area after 7-10 days.

At day 10, two injections of 0.05 ml of IGF-I (50 μg/ml) were made into each lax/sagging upper eye lid. After 3 days, there was a significant reduction in sagging.

### EXAMPLE 2

36 year old female with female pattern hair loss applied 4 ug's IGF1 (0.5 ug/ml) to two sites of scalp bi-weekly for one month and then 4 ug's IGF1 LR bi-monthly with significant increase in new hair growth and increase in diameter of existing hairs.

### EXAMPLE 3

30 year old male was applying subcutaneous and intradermal injections of 2 ug of IGF1 1r IGF1 LR3 to three injection sites along his frontal hair line. He initially was injecting 2 ug of IGF1 LR3 to three injection sites biweekly for a period of two months. He stopped and then intermittently used this same dosing off and on for 1 year. The inventor observed a dramatic reversal of the patient's male pattern balding with re-growth and thickening of the hair along the patient's frontal hair line and a 1-1.5 cm lowering of the frontal hair line.

### EXAMPLE 4

52 year old male with male pattern baldness applied intradermal injections 2 ug of IGF1 LR3 bilaterally into the temporal hair line, a site of extensive miniaturization and recession of the hairline. The bi-weekly injections over six months resulted in reversal of the miniaturization with lengthening, thickening of the hairs and significant advancement of the temporal hair lines.

### EXAMPLE 5

46 year old female with thinning/loss of eyebrows. Using an intradermal injection with a 31 gauge needle, IGF1 2 ug was placed into the eyebrow and was injected weekly for 4 weeks. A significant thickening of the eyebrows and lengthening of eyelashes was noted along with a significant reduction of wrinkles in skin over eyelids and orbit.

### EXAMPLE 6

Chronic Venous Stasis: A 53 year old male displayed venous stasis disease which was secondary to recurrent trauma before and after a crush injury to the right lower leg and ankle 20 years earlier. The patient had developed a dark pigmented rash with progressive pain and swelling in the lower leg and ankle whenever the patient was standing. The patient was initially treated with a single injection with 5 ug of LR IGF1 injected subcutaneously into the center of the rash and the next day the patient experienced a reduction in edema/swelling/pain upon ambulation. As well the inventor noted signs of revascularization with reduced edema, a significant increase in the resistance to edema and pain on prolonged ambulation, and centrally at the site of the initial injection of IGF1 an area of dark blue and black skin became normal in appearance. After two months, a second dose of 4 units of LR IGF1 was injected subcutaneously with even more improvements of the clinical signs and symptoms above of the patients' venous stasis disease of right foot ankle.

### EXAMPLE 7

89 year old female with peripheral vascular disease and arteriosclerotic ischemia induced ulcer of 5 years duration. After the ulcer had been present for approximately 3½ years, the patient underwent intensive conventional medical treatment combined with bi-weekly barometric O2 therapy in a compression chamber for 12 weeks with no noticeable improvement or healing of the ulcer. One year later the patient received monthly subcutaneous IGF1 LR3 injections with 5 ug (1 ug/unit)=5 units total volume injected with a 31 gauge 0.3 ml insulin syringe into the intact skin adjacent to ulcer. The site of the monthly injections was varied around the periphery of the ulcer. Immediate healing and shrinkage was noted within days of the first injection and 90% healing after 3 months. The ulcer had completely healed in 6 months.

### EXAMPLE 8

51 year old male tennis player with partial anterior cruciate ligament tear 12 years earlier with chronic instability of the knee. IGF1 10 g was injected intra-articularly once a month for three doses. After the first injection the patient noted a reduction in pain and swelling after exercising. Physical examination after the third dose revealed reduced luxation indicating increased stability of the anterior cruciate and the patient reported elimination of pain and swelling during tennis and other forms of physical exertion.

### EXAMPLE 9

50 year old female with recurrent tennis elbow. Following 10 ug of IGF1 injected into insertion over site of maximum tenderness ×2 doses a week apart, the patient reported complete elimination of pain and resolution for several months duration.

### EXAMPLE 10

46 year old male with left sided plantar fasciitis. Patient was treated with a single injection into the insertion of the plantar fascia using a dose of 6 ug IGF1. The patient experienced significant reduction in symptoms when he awoke the next day and a complete resolution of pain within 5 days.

### EXAMPLE 11

48 year old male 10K distance runner with bilateral shin splints. Without stopping his running or reducing his daily distance, the patient had two doses of IGF1 10 ug injected subcutaneously with a 30 gauge needle into the site of maximal tenderness for 2 consecutive days to right leg. On the second day the patient reported immediate reduction of pain and the pain elimination within one week. But 2 weeks after the elimination of pain in the right side the patient continued to have symptoms in the left leg. A trial of 0.3 ml of 1% testosterone gel applied twice daily to the shin with significant but only partial improvement over 2 weeks, during which the patient continued running. After two weeks of topical Testosterone to the left shin a single dose of 2 ug IGF1 was given in combination with continued application of the 1% testosterone topical gel with immediate improvement noted within 12 hours. After one more week of topical Testosterone, all treatment was stopped and the patient was asymptomatic.

### EXAMPLE 12

53 year old tennis player with chronic pain and weakness in right and left shoulders secondary to tendonitis (rotator cuff) of supraspinatus and long head of biceps tendon. A single dose was given by subcutaneous injection of 10 ug of IGF1 LR3 over the right tendon with immediate improvement of pain and disability within hours. A second dose was repeated one month later. Patient had significant elimination of pain and increase in strength over the next several months.

### EXAMPLE 13

A 42 year old male with previous hair transplants to cosmetically replace and re-establish a strong frontal hair line 8 years ago at the age of 34. The patient showed significant new hair loss with thinning, miniaturization and hair loss occurring over the vertex and occipital area. 10 ug of IGF1 was applied to two injection sites bi-monthly and the inventor observed a significant thickening and reversal of the hair loss.

### EXAMPLE 14

51 year old male with rare bilateral denervation/paralysis to Flexor Pollicus Longus and the first and second Flexor Profundus Longus muscles from a nerve traction injury to the Anterior Interosseous Nerve. The left Anterior Interosseous Nerve was damaged first with complete paralysis that began to improve after 6 months and after the first 12-18 months there was slight recovery which had then plateaued. So after 3 ½ years there had been no improvement with extreme and persistent weakness and incomplete range of motion without resistance of the corresponding interphallangeal joints. After 2½ years following the onset of the right Anterior Interosseous Nerve traction injury there had been no recovery with persistent and almost complete paralysis of the Flexor Pollicus Longus and the first Flexor Profundus Longus muscles. Injection of 5 units of IGF1 1-2 times a month ×6 injections over flexor compartment/subcutaneous tissue of the proximal flexor compartment of the forearm brought full flexion and range of motion on both hands and normal strength to left hand and from total paralysis to an estimated of 30-50% full power on right. After 1 year there were no further gains and 0.05 ml Deletestryl combined with IGF1 was injected into the right flexor compartment of the forearm with a further noticeable normalization of strength in the right thumb and index finger.

### EXAMPLE 15

51 year old female fell while skiing with a severe instability indicating a severe second and possible full third degree tear to the medial collateral ligament of metacarpal-phallangeal joint of the right thumb. Following biweekly 5 ug IGF1 injections directly into the ruptured tendon, after 4 weeks there was healing and dramatic return in stability, strength and pain free function with a major reduction in recovery time.

### EXAMPLE 16

A 54 year old male was injected bilaterally with 1 ml of 2.56 Grams/ml of Hyaluronic acid. The left side only was premixed with LR3 IGF1 10 ug/ml. The injection site was inferior to infraorbital nerve and into the dermis.

A noticeable reduction in the wrinkles was immediately noticed on both sides, but the left side was noticeably fuller after 1 week, and after 6 months the effect had significantly disappeared on the right side, and though lessened on the left it was still noticeably retained in the left side.

Although this disclosure has described and illustrated certain preferred embodiments of the invention, it is to be understood that the invention is not restricted to those particular embodiments. Rather, the invention includes all embodiments which are a functional or mechanical equivalence of the specific embodiments and features that have been described and illustrated.

1-26. (canceled)

27. A pharmaceutical composition for reducing unwanted cosmetic effects of aging on the skin, to reduce wrinkles, to increase collagen and water content, thicken and increase skin volume and to restore and maintain a youthful appearance to age and sun-damaged skin, for alleviating hair loss, for accelerating revascularization and healing for medical conditions associated with impaired tissue perfusion for a wide range of conditions including venous and arterial vascular diseases, for accelerated healing and recovery for neuromuscular degenerative disorders, or for accelerated healing and repair of tendons, ligaments, or osseous fractures, comprising an effective amount of an insulin-like growth factor selected from the group consisting of IGF-1 (Somatomedin-C) and analogue LR3 IGF1 in admixture with a pharmaceutically-acceptable diluents.

28. A pharmaceutical composition according to claim 27, additionally comprising Hyaluronic acid as a dermal filler.

29. A pharmaceutical composition according to claim 27, additionally comprising a prostaglandin.

30. A pharmaceutical composition according to claim 29, wherein said prostaglandin is selected from prostaglandin E1, prostaglandin E2 and prostaglandin F2.

31. A method of reducing unwanted cosmetic effects of aging on the skin to reduce wrinkles, to increase collagen and water content, thicken and increase skin volume and to restore and maintain a youthful appearance to age and sun damaged skin, or alleviating hair loss, for accelerating revascularization and healing for medical conditions associated with impaired tissue perfusion for a wide range of conditions including venous and arterial vascular diseases, for accelerated healing and recovery for neuromuscular degenerative disorders, or for accelerated healing and repair of tendons, ligaments, osseous fractures, said method comprising administering to a human a composition comprising an effective amount of an insulin-like growth factor selected from the group consisting of IGF-1 (Somatomedin-C) and analogue LR3 IGF1 in admixture with a pharmaceutically-acceptable diluents.

32. A method according to claim 31, wherein said composition additionally comprises a prostaglandin.

33. A method according to claim 32, wherein said prostaglandin is selected from prostaglandin E1 and prostaglandin F2.

34. Use of a pharmaceutical composition comprising an effective amount of insulin-like growth factor selected from the group consisting of IGF-1 (Somatomedin-C) and analogue LR3 1GF1 and a pharmaceutically-acceptable carrier, for reducing unwanted cosmetic effects of aging on the skin to reduce wrinkles, to increase collagen and water content, thicken and increase skin volume and to restore and maintain a youthful appearance to age and sun damaged skin, or alleviating hair loss, for accelerating revascularization and healing for medical conditions associated with impaired tissue perfusion for a wide range of conditions including venous and arterial vascular diseases, for accelerated healing and recovery for neuromuscular degenerative disorders, or for accelerated healing and repair of tendons, ligaments, osseous fractures.

35. A use according to claim 34, wherein said composition additionally comprises a prostaglandin.

36. A use according to claim 35, wherein said prostaglandin is selected from prostaglandin E1 and prostaglandin F1.

37. A method of manufacturing a composition according to claim 27, wherein insulin-like growth factor (IGF) selected from IGF-1 (Somatomedin-C) and LR3 IGF1 is admixed with a pharmaceutically-acceptable carrier.

38. A method according to claim 37, further comprising admixing said IGF with a prostaglandin.

39. A method according to claim 38, wherein said prostaglandin is selected from prostaglandin E1 and prostaglandin F1.

40. A method according to claim 39, wherein said administration comprises subcutaneous injection, high pressure jet device, intracavernous injection, intravenous injection, intramuscular injection, intradermal injection, intradermal implantable slow release device, other medication delivery devices, intra-nasal or topical administration.

41. A method according to claim 40, wherein said administration is topical administration.
